(19) United States
(12) Patent Application Publication
(10) Pub. No.: US 2016/0067102 A1
(43) Pub. Date: Mar. 10, 2016

(54) WOUND DRESSINGS

(71) Applicant: BRIGHTWAKE LIMITED, Nottingham, Nottinghamshire (GB)

(72) Inventor: Stephen COTTON, Nottingham (GB)

(73) Assignee: BRIGHTWAKE LIMITED, Nottingham (GB)

(21) Appl. No.: 14/786,874

(22) PCT Filed: Apr. 24, 2014
(86) PCT No.: PCT/GB2014/051270

§ 371 Date: Oct. 23, 2015

(30) Foreign Application Priority Data
Apr. 26, 2013 (GB) 1307544.5

(51) Int. Cl. A61F 13/00

(52) U.S. Cl. CPC A61F 13/00059; A61F 13/00042; A61F 13/00017; A61F 13/00046; A61F 2013/00153

(57) ABSTRACT

A wound dressing comprises an absorbent body of foam material with first and second major surfaces. The first major surface is, in use, the wound-facing surface of the absorbent body. A thermochromic indicator is incorporated into, or is in intimate contact with, the second major surface of the absorbent body. The thermochromic indicator is sensitive to a change in temperature of the skin below the dressing and so is indicative of an infection.

The present invention relates to wound dressings. More particularly, the present invention relates to foam wound dressings.

Different types of wound dressing are required to meet different clinical needs. However, a common requirement for wound dressings is that they are able to absorb exudate from a wound, while retaining sufficient structure that they can be easily removed from the wound after use without tearing or disintegrating. In addition, infrequent dressing changes are preferable as changing a dressing can aggravate a wound, as well as causing pain and/or discomfort for the patient.

Infection of wounds during the healing process is a major concern in healthcare and, as well as being a source of pain and distress to the patient, can be very expensive. Infection of a wound can greatly increase the length of a patient's stay in hospital. However, preemptive prescribing of antibiotics is not currently recommended due to the rising number of resistant strains of bacteria. It is therefore important to monitor wounds for early signs of infection so that appropriate steps can be taken to treat the infection.

It is known that infection causes the temperature of the skin surrounding a wound to rise. The monitoring of skin temperature under wound dressings to detect infection is known. Methods such as liquid crystal displays and other temperature sensing devices such as thermocouples have been described for use as part of bandages and wound dressings. These allow the wound to be monitored without necessarily having to remove the dressing. As a result of this, the discomfort caused to the patient is reduced.

The general concept of monitoring a wound for signs of infection by incorporating an indicator that is capable of producing a visual indication of temperature at a wound surface in a bandage or wound dressing is known.

For example, in WO2007/144795, a temperature sensing element that is applied to the skin with the aid of a bandage is described. Thermocouples and LED or LCD displays are described as preferred indicators.

Foam dressings are a common form of wound dressing and are known to be of particular use in dressing chronic wounds that have high levels of exudate. Foam dressings are commonly made of polyurethane and comprise open cells that are capable of absorbing large volumes of exudate. It is essential for efficient wound healing that the area of the wound be kept moist but the surrounding skin be kept dry to avoid maceration.

Foams are by nature, however, thermal insulators. It is, therefore, problematic to measure temperature changes of the skin surface that occur under a foam dressing at the non-wound-facing surface of the foam dressing.

Although methods of monitoring the skin surface temperature which measure the temperature at the surface and relay the information to an external indicator such as a thermocouple linked to an appropriate display unit are known and can be used to directly monitor the temperature of the skin below dressings, these types of system are expensive and require the user to be trained in their implementation.

Surprisingly, given the foam's insulating properties, a foam dressing with a simple temperature indicator on the external surface of the dressing has now been devised that enables the temperature of the skin surface covered by the foam dressing to be monitored directly.

According to the first aspect of the invention there is provided a wound dressing comprising an absorbent body of foam material, the absorbent body having first and second major surfaces, the first major surface being, in use, the wound-facing surface of the absorbent body, wherein a thermochromic indicator is incorporated into, or is in intimate contact with, the second major surface of the absorbent body.

The dressing according to the invention is advantageous primarily in that, despite the thermal insulating properties of the foam, the thermochromic indicator on the second (non-wound-facing) surface of the foam absorbent body is sufficiently sensitive to the temperature of the skin below the dressing to allow that temperature to be monitored directly. The thermochromic indicator is capable of indicating a change in temperature of the skin below the foam dressing. Preferably the thermochromic indicator indicates a change in temperature from a temperature that is characteristic of normal, healthy skin to a temperature indicative of infection. This allows the wound to be monitored for signs of infection without removing the dressing, thus providing an early indication of infection, and alleviating a cause of discomfort for patients.

By a “thermochromic indicator” is meant a visual indicator that undergoes a change in response to a change in temperature. For instance, the change may be a change in colour, eg a change from one colour to another, or a change from a colour to colourless, or vice versa.

For a human being, the external skin temperature (ie the temperature measured on the surface of the skin) is normally approximately 32° C. In case of an infection this can rise by several degrees, eg to approximately 37° C.

The thermochromic indicator may be any suitable thermochromic indicator known in the art. Preferably the indicator changes colour at a temperature of between 33 and 39° C. More preferably the indicator changes colour at a temperature of between 35 and 37° C.

In certain embodiments, the thermochromic indicator is a liquid crystal indicator, such as those used in liquid crystal thermometers. The liquid crystal is thermotropic, ie it changes alignment with changing temperature and the wavelength of light reflected by the crystal changes as a result. Typically the liquid crystal will have a cholesteric or twisted nematic structure with a pitch that changes with temperature. A continuous sheet of liquid crystals may be applied to the foam sheet of the dressing of the invention. A continuous sheet of the liquid crystal indicator allows areas of increased temperature to be easily observed by the user. The liquid crystals may be any suitable liquid crystal or combination of liquid crystals known in the art. The liquid crystal may be a mixture of cholesterol derivatives such as cholesteryl nonanoate, cholesteryl oleyl carbonate, cholesteryl pelargonate and cholesteryl benzoate.

In other, currently preferred embodiments, the thermochromic indicator is a thermochromic ink. The thermochromic ink may be any suitable thermochromic ink known in the art. Such inks commonly comprise leuco dyes, the molecules of which are able to exist in two forms, one of which is strongly coloured and the other of which may be colourless. Suitable leuco dyes are those that are transformed from one form to the other by the action of heat. The ink used in the present invention may exhibit an irreversible colour change, but more commonly an ink that exhibits a reversible colour change is appropriate. Suitable inks will be familiar to those skilled in the art and are available commercially, eg from LCR Hallcrest, Riverside Building, Connah's Quay, Flintshire CH5 4DS, United Kingdom or from CTI, 1096 Elkton Drive, Suite 600, Colorado Springs, Colo. 80907, USA.

In currently preferred embodiments, the thermochromic ink becomes colourless at elevated temperature, the loss of colour occurring at a temperature indicative of infection in an underlying wound. In one embodiment the ink changes colour from bright pink to colourless.

In simple embodiments of the invention, a dressing consists of the absorbent body, which in use is applied to a wound and held in place by means of a separate secondary dressing, or simply by means of adhesive tape or the like. In other embodiments, the absorbent body forms part of a composite dressing.

The thermochromic ink may be applied directly to the second major surface of the absorbent body. Preferably, however, the ink is applied to a plastics film that is bonded to the second major surface of the absorbent body. This prevents any possibility of the ink contaminating the wound site, as well as permitting more precise application of the ink. The presence of such a film will also inhibit ingress of bacteria into the wound. The ink may be applied to either surface of the plastics film or, in certain embodiments, the ink may be applied to the interior of the plastics film, for example by double casting the film, ie a layer of the film is cast, the ink is applied to the film and then a second layer of the film is cast on top of the ink. Double casting is also advantageous in that it reduces the risk of there being pinholes present in the plastics film as, although pinholes may be present in each individual casting, the likelihood of any such pinholes being present in both layers of the film and aligned with one another is very low.

The ink may be applied across the full extent of the second major surface. More commonly, however, the ink is applied in a pattern, eg a regular, repeating pattern. The regular pattern makes a temperature change in a small area easier for the user to identify. The pattern may comprise continuous elements, such as lines, or graphic elements arranged in a regular array. For instance, the pattern may comprise an array of dots or other graphic elements. In order to make visible an increase in temperature that occurs in a relatively small region, the spacing between elements in the pattern is preferably less than 10 mm, more preferably less than 5 mm or less than 3 mm.

Printing the ink across the entire absorbent body in a closely repeating pattern is advantageous as it allows the user or medical practitioner to monitor the temperature of the skin at the wound and identify a possible infection. The presence of the pattern across the full extent of the absorbent body prevents false indications of local infection when the temperature of the skin is above a normal temperature over the entire body of the patient, eg as a result of a systemic infection. In the case of a raised temperature across the whole body, the entire pattern will show a change in colour. The continuous pattern also enables the absorbent body to be cut to any suitable size whilst maintaining the ability to monitor the skin temperature.

The thermochromic ink is most preferably applied by a printing process. Suitable printing methods will be familiar to those skilled in the art.

Preferably, the surface of the dressing is, in addition to the thermochromic indicator, printed with another ink that does not change colour with temperature or any other environmental factor. This can be used to print a distinctive pattern across the surface of the dressing which enables the user to identify the dressing as the dressing of the invention if the temperature change has already occurred across the entire surface of the dressing. Thus, in preferred embodiments of the invention, the second major surface of the absorbent body carries not only the thermochromic indicator (for instance a regular pattern of graphical elements printed in thermochromic ink), but also a non-thermochromic visual element (for instance a graphical pattern or image printed in an ink with an appearance that does not change with temperature). The combination of a printed indicator that does change in appearance at elevated temperature and a printed indicator that does not is beneficial in that it differentiates, in use, a dressing according to the invention in which the thermochromic indicator has, for instance, become colourless from a conventional dressing of similar physical form that carries no such indicator.

Indeed, patterns printed on the dressing of the invention in thermochromic and non-thermochromic inks may complement each other, for instance such that a warning symbol or the like printed in non-thermochromic ink may become visible when a surrounding pattern printed in thermochromic ink loses its colour.

The foam material may be any suitable foam known in the art. Usually, the foam is an open-celled foam. Preferably the foam is a hydrophilic foam. More preferably the hydrophilic foam is a polyurethane foam. Most preferably the foam is an open-celled polyurethane foam. The foam typically has a thickness of 0.5 mm to 10 mm, preferably from 1 mm to 7 mm, or from 2 mm to 7 mm, and the foam most preferably has a thickness of 2 mm, 3 mm, 4 mm or 5 mm.

As described above, the foam may be bonded at its second major surface to a plastics film. Preferably the film is a polyurethane film. When the film is present, the thermochromic indicator is applied to the outward-facing surface of the film.

The film will generally be impermeable to wound exudate and other liquids, but is preferably permeable to air and moisture vapour. In particular, the film preferably exhibits a relatively high moisture vapour transmission rate (MVTR). The MVTR of the film may be at least 300 g/m²/24 h, more suitably at least 500 g/m²/24 h and preferably at least 700 g/m²/24 h at 37° C. and 100% to 10% relative humidity difference. The MVTR of film may be up to 2000 g/m²/24 h under such conditions.

It is particularly surprising that the thermochromic indicator is able to provide a reliable indication of increased temperature at the wound site when the foam material has a thickness of 2 mm or more.

Thus, according to a particularly preferred aspect of the invention, there is provided a wound dressing comprising an absorbent body of open-celled foam having first and second major surfaces and a thickness of at least 2 mm, the first major surface being, in use, the wound-facing surface, and the second major surface being bonded to a plastics film that is permeable to air and moisture vapour and which is printed in thermochromic ink with a regular pattern of graphical elements, wherein the thermochromic ink is coloured at ambient temperature, but becomes colourless at an elevated temperature indicative of infection at a wound site to which the dressing is, in use, applied.

As noted above, in addition to the pattern printed in thermochromic ink, the dressing may be printed with a non-thermochromic pattern that remains visible when the thermochromic ink becomes colourless.

Where the absorbent body forms part of a composite dressing, it will generally be overlaid with a backing layer, which forms a barrier between the wound and the surrounding atmosphere and may also extend beyond the absorbent body to provide a means of attachment of the dressing to the skin around the wound site.

The backing layer is most preferably a plastics film, eg a polyurethane film, having the desired characteristics, which may be similar to those described above in relation to the plastics film that may be bonded to the foam material.

The backing layer may be transparent, in which case the thermochromic indicator may be applied to the second major surface of the absorbent body (eg directly to the foam material or to a plastics film bonded to the foam material as described above) such that it can be seen through the backing layer.

Alternatively, the thermochromic indicator may be applied to the backing layer itself, which is bonded to the absorbent body. In such a case, the backing layer does not need to be transparent.

The backing layer may be larger in size than the absorbent body, such that it extends beyond the edge of the absorbent body on one or more sides. Preferably, the backing layer extends beyond the edge of the absorbent body on all sides, forming a border around the absorbent body.

Where the backing layer forms a border around the absorbent body, the border may carry an adhesive to adhere the wound dressing to the patient's skin around the wound. Suitable skin contact adhesives for wound dressings are known, and any suitable adhesive known in the art may be used in the present invention.

Where the wound dressing includes a skin contact adhesive, the dressing will generally be supplied with a releasable liner on its underside. The releasable liner may cover the adhesive and wound contact portions of the wound dressing prior to use, and be removed from the dressing immediately before application of the dressing to the wound. This reduces the risk of contamination of the wound dressing and facilitates handling of the dressing.

The invention will now be described in greater detail, by way of example only, with reference to the accompanying drawings, in which

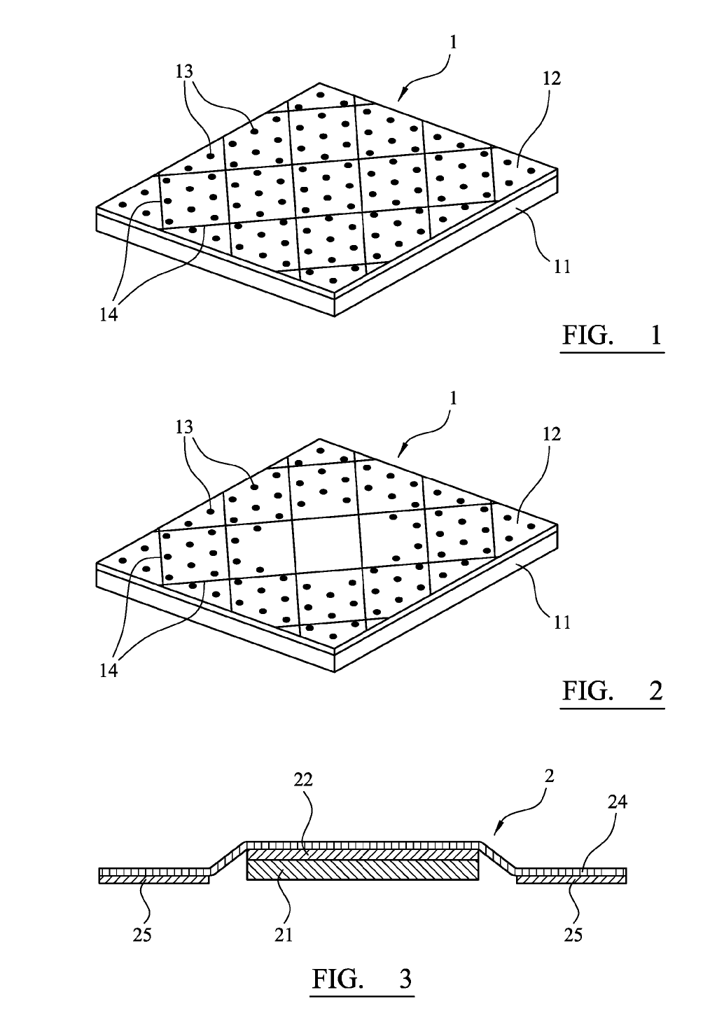

FIG. 1 shows a perspective view of a first embodiment of a wound dressing according to the invention;

FIG. 2 is a view similar to FIG. 1, but showing the appearance of the dressing in the event of a rise in temperature below it, for instance where the wound to which it is applied becomes infected; and

FIG. 3 shows a cross-sectional view of a second embodiment of a wound dressing according to the invention.

In FIG. 1 there is shown a first embodiment of a wound dressing according to the invention, generally designated 1. The dressing 1 is generally square in form and comprises an absorbent body of open-celled polyurethane foam 11, to the upper (as viewed in FIG. 1) surface of which is bonded a breathable polyurethane film 12. The foam sheet is approximately 5 mm thick.

A regular pattern of dots 13 is printed on the film 12 using a thermochromic ink. In addition, the film 12 is printed with a pattern of diagonal lines 14 in non-thermochromic ink.

In use, the dressing 1 is applied to an exuding wound, eg an ulcerous wound such as a leg ulcer. The dressing 1 is secured in place by means of a secondary dressing, eg an occlusive film dressing, or simply by means of adhesive tapes. Exudate is absorbed by the foam 11, liquid material being retained within the foam 11 by the polyurethane film 12, though air and water vapour can pass through the film 12 by virtue of its breathability.

The thermochromic ink used for the printing of the pattern of dots 13 changes from a strong visible colour at ambient temperature to colourless at more elevated temperature, and specifically at a temperature reached in the event that the wound site beneath the dressing becomes infected. This leads to a change in the appearance of the dressing as shown in FIG. 2, where it can be seen that the dots near the centre of the dressing 1 have lost their colour. This indicates to the patient, or to a responsible healthcare professional, that there has been a local rise in temperature beneath the dressing, which may be due to infection of the wound. The diagonal lines 14 are not affected by the increase in temperature and so remain visible across the full extent of the dressing 1.

Of course, in the event of a more general infection resulting in a fever or elevated systemic temperature, the whole pattern of dots 13 may disappear, which (at least where the dressing 1 is substantially greater in extent than the underlying wound site) allows for differentiation between a local infection and a more general, systemic infection. Again, in such a case, the diagonal lines 14 are unaffected and remain visible.

A second embodiment of a wound dressing, generally designated 2, is shown in FIG. 3. This dressing 2 incorporates an absorbent body that is broadly similar to the dressing 1 of FIG. 1, and which comprises open-celled polyurethane foam 21 to which is bonded a polyurethane film 22. The film 22 is printed in thermochromic ink with a pattern of dots (not visible in FIG. 3) similar to that present on the dressing 1, and with non-thermochromic diagonal lines, as for the first embodiment.

In the dressing of FIG. 3, however, the upper (as viewed in FIG. 3) surface of the absorbent body is overlaid by, and adhered to, a backing layer 24 comprising a sheet of transparent microporous polyurethane. The backing layer 24 extends beyond the foam 21 in all directions such that it forms a border around the foam 21. The underside of that border carries a layer of skin contact adhesive 25, by which the dressing may be affixed to the healthy skin surrounding a wound site.

Because the backing layer is transparent, the pattern of dots applied to the film 22 is visible through it, as are the diagonal lines printed in non-thermochromic ink. In a similar manner to the dressing 1 of FIG. 1, therefore, the disappearance of the dots provides an indication of a local infection or (in the case of complete disappearance of the pattern of dots across the whole extent of the absorbent body) of a systemic infection.

In other embodiments, the patterns of dots and lines or other pattern may be applied to the upper surface of the backing layer 24, in which case the backing layer 24 does not need to be transparent and the polyurethane film 22 may be omitted, the foam 21 being affixed directly to the backing layer 24.

1. A wound dressing comprising an absorbent body of foam material, the absorbent body having first and second major surfaces, the first major surface being, in use, the wound-facing surface of the absorbent body, wherein a thermochromic indicator is incorporated into, or is in intimate contact with, the second major surface of the absorbent body.

2. A dressing according to claim 1, wherein the thermochromic indicator indicates a change in temperature from a temperature that is characteristic of a normal, healthy skin to a temperature indicative of infection.

3. A dressing according to claim 1, wherein the change in temperature indicated by the indicator is from a temperature that is characteristic of a normal, healthy skin to between 33° and 39° C.

4. A dressing according to claim 3, wherein the change in temperature indicated by the indicator is from a temperature that is characteristic of a normal, healthy skin to between 35° and 37° C.

5. A dressing according to claim 1, wherein the indicator is a thermochromic ink.

6. A dressing according to claim 5, wherein the ink is applied in a regular pattern.

7. A dressing according to claim 6, wherein the spacing between elements in the pattern is less than 10 mm.

8. A dressing according to claim 7, wherein the spacing between the elements is less than 5 mm.

9. A dressing according to claim 8, wherein the spacing between the elements is less than 3 mm.

10. A dressing according to claim 5, wherein the ink is applied by a printing process.

11. A dressing according to claim 5, wherein the ink is coloured at ambient temperature and colourless at an elevated temperature.

12. A dressing according to claim 1, wherein, in addition to the thermochromic indicator, a non-thermochromic visual element is incorporated into, or is in intimate contact with, the second major surface of the absorbent body.

13. A dressing according to claim 1, wherein the foam is an open-celled foam.

14. A dressing according to claim 1, wherein the foam is a hydrophilic foam.

15. A dressing according to claim 1, wherein the foam is a polyurethane foam.

16. A dressing according to claim 15, wherein the foam is an open-celled polyurethane foam.

17. A dressing according to claim 1, wherein the foam has a thickness of 0.5 mm to 10 mm.

18. A dressing according to claim 17, wherein the foam has a thickness of from 1 mm to 7 mm.

19. A dressing according to claim 18, wherein the foam has a thickness of from 2 mm to 7 mm.

20. A dressing according to claim 1, wherein a film is bonded to the second major surface of the absorbent body.

21. A dressing according to claim 20, wherein the film is a plastics film.

22. A dressing according to claim 21, wherein the film is a polyurethane film.

23. A dressing according to claim 20, wherein the film is permeable to air and moisture vapour.

24. A dressing according to claim 23, wherein the moisture vapour transmission rate of the film is at least 300 g/m²/24 h.

25. A dressing according to claim 20, wherein the thermochromic indicator is applied to the film.

26. A dressing according to claim 25, wherein the thermochromic indicator is a thermochromic ink applied to the film in a regular pattern of graphical elements.

27. A dressing according to claim 1, wherein the dressing comprises an absorbent body of open-celled foam having first and second major surfaces and a thickness of at least 2 mm, the first major surface being, in use, the wound-facing surface, and the second major surface being bonded to a plastics film that is permeable to air and moisture vapour and which is printed in thermochromic ink with a regular pattern of graphical elements, wherein the thermochromic ink is coloured at ambient temperature, but becomes colourless at an elevated temperature indicative of infection at a wound site to which the dressing is, in use, applied.

28. A dressing according to claim 27, wherein the plastics film is further printed with a non-thermochromic pattern that remains visible when the thermochromic ink becomes colourless.
